(19) United States
(12) Patent Application Publication
(10) Pub. No.: US 2018/0361090 A1
(43) Pub. Date: Dec. 20, 2018

(54) MEDICAL DEVICE FOR DELIVERY OF FLUID TO A PATIENT

(71) Applicant: Southmedic Incorporated, Barrie (CA)

(72) Inventors: Lee McDonald, Barrie, Ontario (CA); Alex McDonald, Barrie, Ontario (CA); Andy Morum, Barrie, Ontario (CA); Robert Burke, Barrie, Ontario (CA)

(21) Appl. No.: 15/777,464

(22) PCT Filed: Nov. 18, 2016
(86) PCT No.: PCT/CA2016/051353

§ 371 Date: May 18, 2018

Related U.S. Application Data
(60) Provisional application No. 62/257,417, filed on Nov. 19, 2015.

(51) Int. Cl. A61M 16/00; A61M 16/10; G16H 40/63; G16H 50/30

(52) U.S. Cl. CPC A61M 16/024; A61M 16/1005; G16H 40/63; G16H 50/30; A61M 2230/432; A61M 2205/3569; A61M 2205/3592; A61M 2205/6018; A61M 2230/205; A61M 2202/0208

(57) ABSTRACT

We disclose an apparatus, system, and method for controlling a level of a compound in a patient with a combination of a fluid administration device, a sensor and a controller. The sensor detects the level of a compound within the patient and transmits this data relating the controller. The controller controls fluid flow rate to the device. A unit is provided that connects to the device, enabling the device to operate in a “smart device” mode such that the device effectively communicates with the controller. The unit includes a data storage unit for storing digital information identifying the fluid delivery device and/or properties thereof, and a data transmitting unit for electronically transmitting the stored information in the data storage unit to the controller.

### CROSS REFERENCE TO RELATED APPLICATION

This application claims Paris Convention priority to U.S. application No. 62/257,417, filed on Nov. 19, 2015, and which is incorporated herein by reference, in its entirety.

### TECHNICAL FIELD

The present application relates to medical devices, in particular systems for delivering a fluid such as oxygen to a patient.

### BACKGROUND

Medical devices may administer a breathable gas such as oxygen or other fluid to a patient via a mask, cannula or the like. The rate of delivery of the fluid is normally dictated in part by the parameters of the delivery device, which is normally configured to operate efficiently within a specific range of flow rates. Within this range, a more specific rate may be determined by patient-based parameters such as the blood level within the patient of a specified compound such as oxygen. For example, for administration of a compound that enters the patient's blood, if the patient's blood level of the compound drops below a certain level, it can be desirable to increase the flow rate.

There exist various means in the art to measure the blood level of various compounds within the patient. In the case of oxygen, the patient's blood oxygen level can be detected in a non-invasive fashion with a pulse oximeter. Such means can detect the circulating level of a compound within the patient on a real time, or near-instantaneous fashion, and transmit this information electronically on a real time or periodic basis.

Typically, when oxygen is supplied to a patient, a healthcare practitioner sets the oxygen delivery rate manually. As the level of arterial oxygen saturation in the patient changes, it may be necessary or desirable to adjust the gas flow rate. It can be difficult to manually monitor the patient's blood oxygen level with sufficient attention and respond quickly to changes in this level with adjustments to the gas delivery rate. The same issues can arise with respect to administration of other compounds such as a nebulized drug.

A further background aspect relates to the fact that different configurations and types of fluid delivery devices typically provide different fluid delivery efficiencies. For example, different oxygen masks can have different levels of efficiency whereby a lower flow rate may be required in one mask than another type of mask to provide the same level of oxygen saturation in a patient. This factor can make it difficult for the health care provider to adjust the gas flow rate for any particular device, especially when a number of different types of devices are used by the institution.

Turning to previously-published background information, U.S. Pat. No. 8,777,894 describes a data communication system used for administering IV fluids.

### SUMMARY

According to one general aspect, we disclose a system for administering a fluid to a patient, wherein the system receives real-time data of the level of one or more compounds within the patient (such as blood oxygen level) and automatically adjusts the fluid flow rate that is delivered to the patient in response to a combination of the detected levels of the compound in the patient and parameters of an associated fluid delivery device. The compound detected within the patient may be the same as or different from the compound administered to the patient.

According to a further aspect, we disclose an apparatus for communicating between a medical device, a sensor and a fluid delivery controller. The medical device is for delivering a fluid to a patient, the sensor is for detecting the level of at least one compound within a patient and transmitting data relating to this level to the controller, and the controller is adapted to control fluid flow rate to the medical device. According to this aspect, the apparatus comprises:
a body configured for connection to said device;
a data storage unit for storing digital information identifying said fluid delivery device and/or properties thereof (for example, an operational flow rate range of said medical device); and a data transmitting unit for electronically transmitting the stored information in the data storage unit to the controller.

The sensor detects the level of a compound in the patient and converts this information into an electronic signal. The detection and conversion steps of the sensor may be performed within a single component or in separate components. For example, an oximeter may comprise a first unit that measures light transmission through a portion of the patient's body (such as ear lope or finger) and transmits the detected light level to a second unit that translates this information to a blood oxygen level. In the case of CO2 measurement, the delivery device may include a breath collector incorporated within an oxygen mask, connected by a tube to a unit that measures the CO2 concentration in the exhaled breath and generates data indicative of this level. In both such cases, the data processing functions may be performed remotely from the sample acquisition functions, whereby the data processing is performed within the controller described herein or in a unit that is separate from the controller.

In one embodiment, the body of the present apparatus comprises a conduit having a first end for direct or indirect connection to the controller, an opposing second end for direct or indirect connection to the medical device and a bore between the first and second ends for fluid flow through the conduit to the medical device.

The body may be releasably connectable to the medical device or integrated with the medical device.

The transmitting unit may automatically transmit the stored information when the apparatus is operatively connected to the medical device and/or a source of the fluid.

The data storage unit and the transmitting unit may be integrated within an RFID tag.

We also disclose a system for controlling a fluid flow rate delivering to a patient through a medical device, comprising a control unit comprising a controller and a fluid regulator for controlling the flow rate of a fluid suitable for administration to a patient in response to electronic signals from said controller; a data storage unit and a data transmitting unit as described above. The controller is configured to receive electronic signals from said data transmitting unit and from a detector that is adapted to be worn or otherwise placed in operative association with a patient to detect a level of a selected compound within said patient and to transmit a signal to said controller indicative of the detected level of said compound, wherein said controller is configured to process signals with a feedback loop to provide a recursive control over the delivery rate of said fluid to bring or maintain the level of said compound within the patient within a predetermined range or predetermined level.

We also disclose a method for controlling the level of a compound in a patient, wherein the level of the compound is responsive to a fluid administered to the patient through a medical device. The method comprises the steps of:
receiving from a data transmission unit, by a controller, a first data identifying said device and/or properties thereof;
receiving from a sensor, by the controller, a second data indicating the level of the compound in the patient; and
in response to the first data and the second data, transmitting to a fluid regulator, by the controller, at least one command for the fluid regulator to generate a fluid flow rate to be administered to the patient through the device, wherein the fluid flow rate generated by the fluid regulator is within a range determined by the first data. The method may comprise use of the apparatus or the system described herein.

According to a still further aspect, we describe a method for selecting a medical device in response to patient response to fluid delivery. The method comprises:
transmitting, from a connector, to a controller device identification and/or device parameters relating to the device;
in response to the device identification and/or device parameters, generating a maximum fluid flow rate and a minimum fluid flow rate supported by the device;
receiving from a sensor, by the controller, a first compound level in the blood of the patient in response to the maximum fluid flow rate, and a second compound level in the blood of the patient in response to the minimum fluid flow rate;
comparing, by the controller, the first compound level and the second compound level to a target SpO2 level, respectively; and
selecting the device, when the first compound level is equal to or greater than the target compound level, and the second compound level is equal to or less than the target compound level.

### BRIEF DESCRIPTION OF THE DRAWINGS

FIG. 1 is a block diagram of a gas delivery system according to an embodiment of the invention.

FIG. 2A is a perspective view of a connector according to the system of FIG. 1.

FIG. 2B is a perspective view of an example kit containing a connector according to the system of FIG. 1 and a plurality of delivery devices.

FIGS. 3A and 3B provide a table showing flow rate ranges of selected fluid delivery devices.

FIGS. 4A and 4B depict a flow chart showing a method for selecting a fluid delivery device according to a further embodiment of the invention, in which FIG. 4B continues of the flow chart of FIG. 4A.

FIG. 5 is a flow chart showing a method for regulating fluid flow rate with a selected type of fluid delivery device according to a further embodiment.

### DETAILED DESCRIPTION

System 100 receives a pressurized fluid to a fluid delivery device 114 for administration of the fluid to a patient. Suitable fluids are those that can be administered to a patient in a rate-controlled fashion with a device, and which contain a compound that can be quickly detected and measured in the patient's body. Suitable fluids include both liquids and gasses, with a particularly suitable gas being oxygen. A nebulized drug can also be a suitable fluid. The term “gas” as used herein refers to a substance which is in the gas phase when under conditions of normal ambient temperature and within a pressure range experienced during routine patient care. The term “oxygen” as used herein represents an example of a suitable fluid, and can refer to pure or substantially pure oxygen or oxygen-enriched air. Fluid source 104 can include, for example, a hospital-wide source of oxygen, or alternatively any other fluid that is normally administered to a patient in a controlled, rate-limited fashion through a fluid delivery device. The term “operational fluid flow rate range” of fluid delivery device refers to a fluid flow rate range supported by the fluid delivery device with the delivery device working properly.

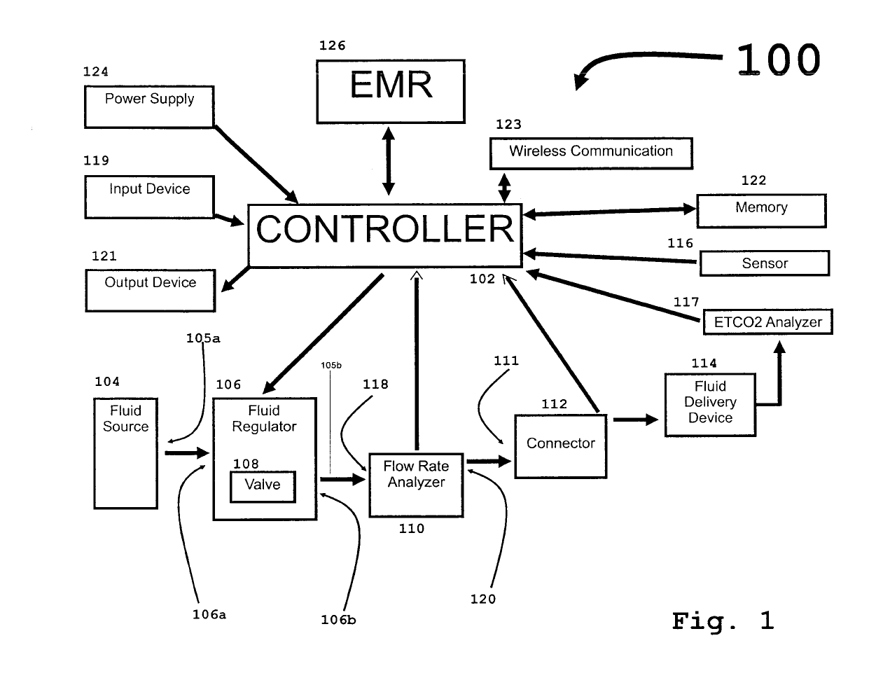

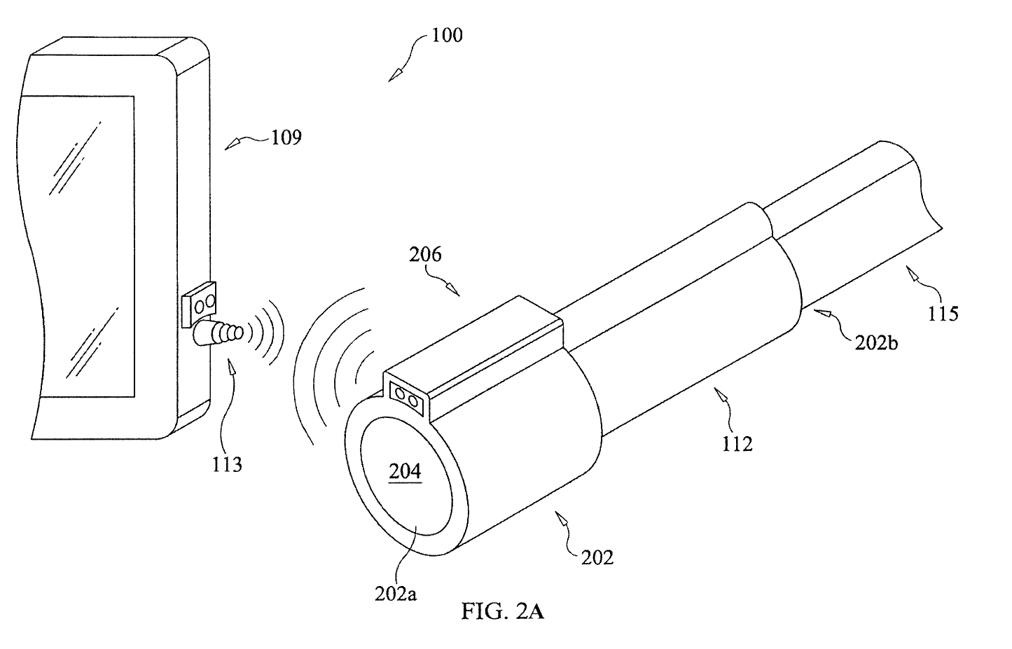

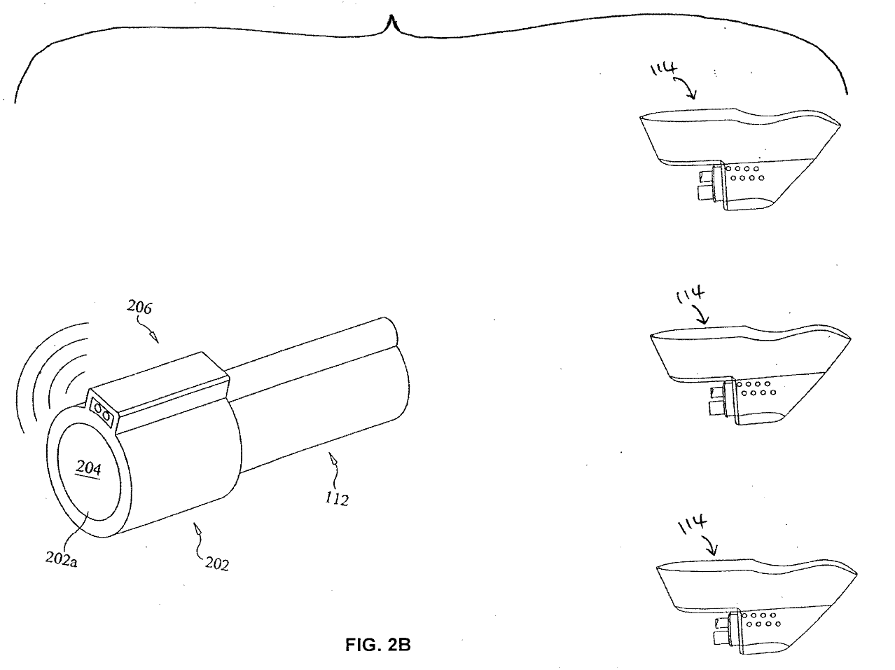

A fluid is delivered to system 100 from a fluid source 104. Referring to FIGS. 1 and 2, system 100 comprises a housing 109 (seen in FIG. 2A) which houses a controller 102 and other components. The controller may be a processor for processing information received or generating commands to other components. Housing 109 may support external display and control components, not shown.

System 100 is herein described, by way of example and for ease of understanding, in terms of a gas flow sequence. According to this aspect, pressurized gas is delivered from an external source 104 and ultimately administered to a patient via a medical device 114 such as an oxygen mask or other device for delivering a fluid to a patient.

Concurrently operating with the fluid flow sequence, system 100 provides a digital information flow sequence which serves to automatically regulate the gas flow delivered by the system 100. In particular, gas flow to the patient is controlled by system 100 in response to information and data received from a connector 112 connected to the delivery device 114 and a digital information gathering component 116 worn by the patient. The gas flow components are connected together via various connection means that provide leak-proof connection using conventional means such as appropriate tubing, gaskets, threaded or push-in connectors and the like.

System 100 accommodates various types and models of delivery devices 114. These may differ in their parameters and properties such as different operational gas flow rates, efficiency levels of gas delivery etc. For purposes of describing the present embodiment, a representative delivery device 114 is described, which is sometimes referred to herein as “the selected delivery device 114”. In use, as described below, device 114 may be selected based on various parameters and properties of the device that optimize the selected device for a particular patient application.

Turning first to the fluid flow aspect, shown schematically in FIG. 1, system 100 includes a fluid flow regulator 106 that receives pressurized fluid from source 104 and controls the flow rate of the fluid to device 114. Regulator 106 is housed within housing 109. Regulator 106 is responsive to control signals from controller 102. Fluid from external source 104 may enter regulator 106 via a conventional connector, not shown, that may be integral with housing 109. Gas flows from fluid source 104 to input end 106a of fluid regulator 106, directly or via a fluid conduit 105a. Fluid regulator 106 may comprise an electronically controllable valve 108, such as (for example) a piezoelectric valve or other fluid regulator known to the art. Regulator 106 is responsive to electronic commands from controller 102 to vary the gas flow rate exiting regulator 106. One such command can set an initial flow rate of gas. Fluid regulator 106 has a fluid output 106b to discharge fluid from regulator 106.

Next in line is optionally a flow rate analyzer 110 which receives fluid discharged from output 106b of fluid regulator 106, optionally through a conduit 105b. Analyzer 110 comprises a gas input 118 and an output 120. Analyzer 110 comprises a device of a type known to the art, to measure the flow rate and optionally the pressure of fluid discharged from regulator 106. Analyzer 110 transmits this flow rate data to controller 102 in electronic digital form. Gas exiting output 120 is then discharged from housing 109 via a connector 111, such as a nozzle.

Next in line, external to housing 109, is a connector 112 that operatively connects fluid delivery device 114 to fluid discharged from the flow rate analyzer 110. Connector 112 is releasably connected to nozzle 111 of housing 109 to receive gas discharged from analyzer 110. Connector 112 incorporates electronic components, for example within body 202, for storing and transmitting information in digital, electronic form relating to specific parameters of fluid delivery device 114, as discussed in more detail below.

Furthermore, as discussed below, connector 112 represents an embodiment of a unit that enables the delivery device 114 to operate in a “smart” mode whereby the device effectively communicates with controller 102 in conjunction with a patient sensor 116, also discussed below.

Next in line is medical device 114, which is operatively connected to connector 112. In this example, device 114 comprises a generally conventional oxygen delivery mask, nose cannula or other device for delivering a fluid to a patient in a controlled fashion. For example, device 114 may be an OxyMask™ gas delivery device by Southmedic, Inc. Device 114 may connect to connector 112 via a flexible tube. Alternatively, connector 112 connects directly to device 114 whereby the connecting tube is located between connector 112 and housing 109.

Device 114 normally is configured to administer fluid to a patient within an optimal range of flow rates, whereby operating outside this range is either inefficient or otherwise provides medically undesirable outcomes.

It will be appreciated that the electronic components of connector 112 may instead be integrated within delivery device 114 whereby a discrete connector component is not required. Alternatively, the electronic components of connector 112 may be housed within a body that can be secured to or otherwise associated with device 114.

Controller 102 communicates electronically with fluid regulator 106 to adjust the gas flow rate in response to electronic data inputs that controller 102 receives from multiple sources. The first such source comprises flow rate analyzer 110, whereby the input to controller 102 is indicative of the flow rate of gas delivered to device 114. For “steady state” operation of system 100, controller 102 compares the delivered flow rate, in real time, with the initial flow rate contained in a command sent to fluid regulator 106. If the detected flow rate differs from the predetermined, selected flow rate, controller 102 commands fluid regulator 106 to adjust valve 108 to increase or decrease the flow rate until the flow rate detected by analyzer 110 matches or substantially matches the selected flow rate. This control step is performed in an iterative process until the detected flow rate matches or substantially matches the initial flow rate. As such, controller 102, fluid regulator 108 and flow rate analyzer 110 provide a feedback loop to maintain a steady rate of fluid delivery even if flow rates or pressures from source 104 vary.

The second input to controller 102 may consists of electronic digital signals received from one or more sensors 116, 117. In the present example, sensor 116 detects oxygen saturation in the patient and sensor 117 detects breath CO2 concentration. Oxygen and CO2 sensors 116 and 117 may both be used or only one such sensor may be used. It will be seen that any suitable sensor or sensors may be provided for detecting the level of one or more compounds within the patient, preferably in a non-invasive fashion. Sensors 116, 117 detect one or more compounds which may be naturally occurring compounds such as oxygen, administered substances such as a drug or a by-product such as a drug breakdown product. The present example is described first by reference to sensor 116, which is a pulse oximeter that non-invasively detects blood oxygen levels and can transmit this information to controller 102. Another example is a sensor 117 for detecting CO2 concentration in the patient's exhaled breath. Other suitable sensors detect other compounds in the patient's bloodstream, skin or otherwise.

Sensor 116 can operate on a continuous basis whereby data is continuously collected from the patient and transmitted to controller 102 in real time or alternatively (and less preferably) detects compound levels on a periodic basis. Sensor 116 collects the compound information and transmits this information to controller 102, either wirelessly or through a cable or the like.

Sensor 116 may perform the entirety of its functions within a single, integral unit. For example, sensor 116 may comprise a pulse oximeter that transmits light through a body part of the patient, converts the detected light properties into an oxygen saturation level and transmits this information to controller 102 as an electronic signal. Alternatively, some of the functions of sensor 116 may be performed remotely from the unit that is in contact with the patient. For example, an oximeter may transmit detected light properties to controller 102, which in turn converts this information into an oxygen saturation level.

In another example, one of the sensors is an End Tidal Carbon Dioxide concentration (ETCO2) analyzer 117 (see FIG. 2). According to this example, the patient's breath is captured by a breathing mask, not shown. Exhaled breath flows through a tube 119 to ETCO2 analyzer 117 for measuring CO2 content in the patient's breath. Analyzer 117 may be housed in housing 109 whereby tube 109 may be attachable to housing 109 via conventional removable attachment means. ETCO2 analyzer 117 may then electronically transmit the measured ETCO2 value to controller 102, either wirelessly or through a cable or the like. Alternatively, analyzer 117 may be external to housing 109; in such as case, the electronic communications with controller 102 are performed in a similar fashion.

Controller 102 receives data from oxygen sensor 116 and/or from ETCO2 analyzer 11 to regulate gas flow to the patient in response to this data. In this fashion, oxygen administration is regulated in response to the patient's blood oxygen level, and/or ETCO2 level, in a recursive, feedback loop fashion to maintain or achieve a selected blood oxygen level and/or CO2 breath level in the patient. This aspect is described in more detail below.

The third input to controller 102 is from connector 112. The controller 102 may control flow rate delivered by the delivery device 114 in response to receiving information specific to delivery device 114.

Controller 102 integrates the three inputs identified above to control regulator 106 to deliver gas at a rate optimized to achieve or maintain a selected level of a specified substance such as oxygen in the patient (in one example), as will be discussed in more detail below.

Controller 102, regulator 106, analyzer 110 and associated electronic and gas flow components are housed within housing 109, seen in FIG. 2. In another embodiment, ETCO2 analyzer 117 may also be housed within housing 109. Housing 109 includes conventional external connectors, not shown, to connect with one or more fluid input lines and an electrical power source. Alternatively, system 100 may be powered by internally-housed batteries. Housing 109 also comprises gas output nozzle connector 111, which is configured to mate with connector 112. Connectors 111 and 112 may comprise conventional leak-proof, releasable connection means such as a projecting hollow prong/socket arrangement as seen in FIG. 2. Housing 109 may provide the male connector component and connector 112 may provide the female component or vice versa. Housing 109 may also comprise an external or internal receiver 113, shown in FIG. 2A as an external receiver. Receiver 113 receives information or signals carrying the information from a data transmission unit 208 of connector 112 as described below, and transmits the received information to controller 102 whereby controller 102 may determine the flow rate range supported by delivery device 114 that is or may be connected with the connector 112 and may adjust the gas flow rate accordingly.

In one embodiment, connectors 111 and 112 are configured to provide a unique, specifically configured connection arrangement whereby connector 112 is not normally connectable to other, conventional gas sources and likewise connector 111 of housing 109 is not normally connectable to conventional delivery devices. In another embodiment, connectors 111 and 112 are configured to provide conventional, “industry standard” connection means whereby connector 112 may be connected to a conventional gas source that does not provide a control system 100 and, on the other hand, system 100 may if desired connect to a conventional delivery device.

According to the embodiment shown in FIG. 2A, connector 112 comprises a body 202 having a fluid inlet opening 202a that mates to housing connector 111 and a fluid outlet opening 202b that leads to fluid delivery device 114. A bore 204 extends between openings 202a and b for fluid flow. Connector 112 may be integral with a gas tube 115 leading to delivery device 114 or alternatively, connector 112 may be releasably connectable to gas tube 115 via a conventional connection means, not shown. In the case of integral connection, the delivery device 114 and connector 112 would normally be supplied to the user as a unit. In the case of a removable connector, delivery device 114 and connector 112 can be supplied the user separately or as a kit composed of one or more connectors 112 and multiple delivery devices 114, as shown in FIG. 2B. Since connector 112 is normally pre-programmed to store information relating to the parameters of a selected delivery device 114, such a kit would normally consist of multiple units of delivery devices 114.

Connector 112 further comprises a data storage unit 206 and a data transmission unit 208 for storing and transmitting information in digital electronic form. In an example, the data storage unit 206 and data transmission unit 208 may be a Radio-Frequency Identification (RFID) chip embedded within connector 112.

After an electronic “handshake” is made between the connector 112 and the controller 102, the information related to the delivery device 114 and stored in the data unit 206 is transmitted to the controller 102. When connector 112 is connected with system 100, for example, with nozzle connector 111 of the flow rate analyzer 110, connector 112 transmits to controller 102 information stored in connector 112. The information includes certain parameters specific to delivery device 114 that is connected with the connector 112 or may be connected with the connector 112 for delivering fluid to a patient.

In one embodiment, the information received by the controller 102 may be the type or identity of delivery device 114, from which the controller may further determine the specific flow rate supported by the delivery device 114 based on information pre-stored in the controller 102. For example, data unit 206 stores the identifier or type of the delivery device 114 that is or may be connected with the connector 102. The relevant device parameters and other pertinent information relating to device 114 is stored within system 100, for example, in the memory 122, and controller 102 may retrieve relevant parameters or pertinent information of the device 114 from the memory 122 based on the identifier or type of the delivery device 114 received from the connector 102. According to this embodiment, the identifier identifies device 114 with a code that is specific to the individual device 114 and which identifies to controller 102 the specific device 114.

In this embodiment, controller 102 is pre-programmed with the parameters of multiple devices 114 to allow it to control a variety of such devices in response to the specific “handshake” it receives. In one aspect, controller 102 is pre-programmed with parameters of multiple delivery devices 114, and in that case, the information stored in data storage unit 206 of connector 112, and transmitted to controller 102, is indicative of the parameters of selected device 114. In other cases, data storage unit 206 may be pre-programmed to store information that provides specific parameters of a single selected delivery device 114 such as its operational flow rate range or other parameters specific to the specific selected device 114.

In another embodiment, data unit 206 of connector 112 stores with selected information relating to device 114. This information is then transmitted to controller 102, which then may adjust fluid flow rate in response to the parameters received from connector 112. For example, the information received by the controller 102 may include the specific flow rate supported by the delivery device 114. In this embodiment, controller 102 is agnostic as to the device 114 in that it need not be pre-programmed for any specific devices. Rather, controller 102 responds the parameters transmitted to is by connector 112.

With either of the above embodiments, the information transmitted to controller 102 signals either the identity of delivery device 114 that is or may be connected to connector 112 or its specific parameters, whereby controller 102 can determine the desired gas flow rate based on parameters of delivery device 114.

In one embodiment, connector 112 is integrated with device 114 (for example, permanently affixed to its gas tube). In this embodiment, connector 112 is programmed to store in data unit 206 information related to delivery device 114, such as flow rate supported by delivery device 114.

In another embodiment, as shown in the example of FIG. 2B, connector 112 is removable from delivery device 114 and may be supplied independently. For example, connector 112 may be non-disposable, for temporary attachment to a disposable device 114. In this embodiment, connector 112 may be visually marked (for example by colour) to match a similar visual marking in device 114. The kit may include at least one delivery device 114, for example, three delivery devices 114 in the example of FIG. 2B.

In a still further embodiment, the electronic components of connector 112 (data storage unit 206 and data transmission unit 208, or RFID chip) may be integral with device 114. In a still further embodiment, these electronic components may be housed in a body that does not serve a connector function, that may be secured to device 114 or otherwise used in association with device 114 to transmit the information related to delivery device 114 to controller 102 when device 114 is attached thereto.

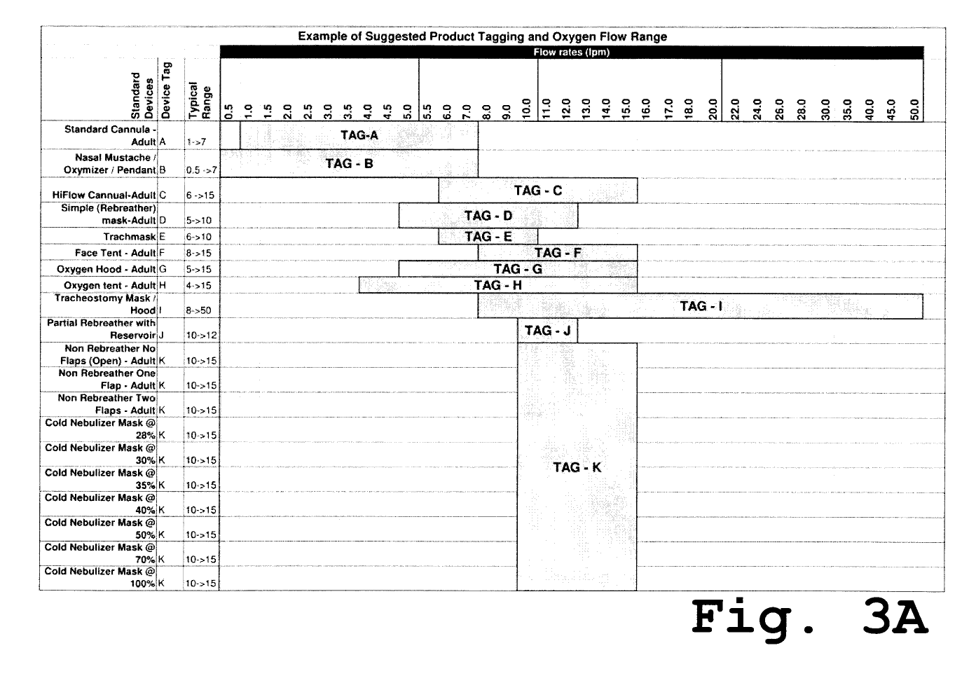

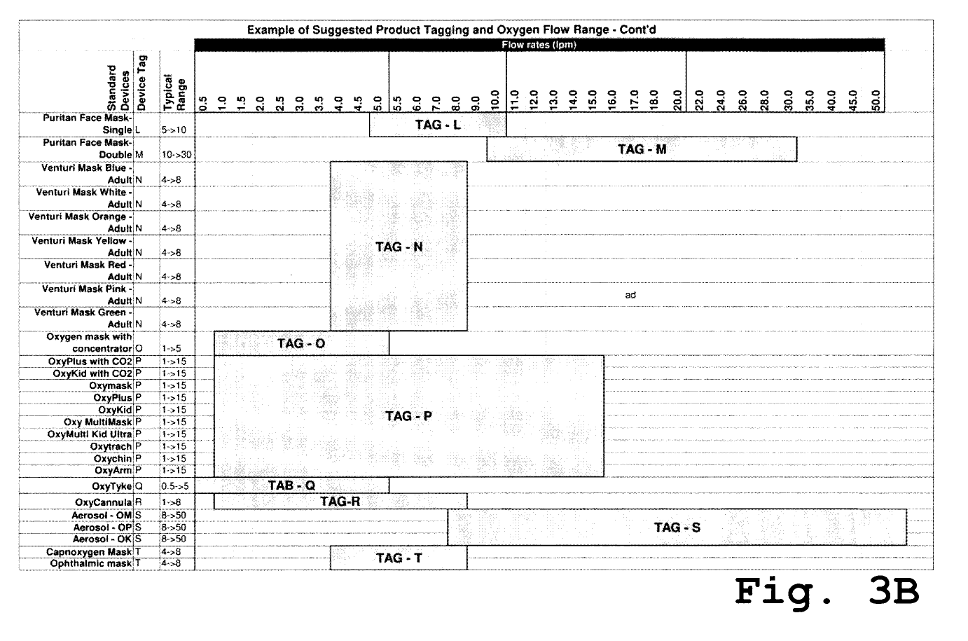

As discussed above, in one embodiment data storage unit 206 stores information unique to a selected delivery device 114. In one embodiment, controller 102 is adapted to operate with a variety of different delivery devices 114 having different operational fluid flow rate ranges. In this aspect, different connectors 112 provide different identifier information to controller 102 to permit controller 102 to adjust fluid flow rates in a manner that is responsive to specific parameters of different delivery devices 114. By way of example, several different types of fluid delivery devices 114 and their corresponding operational flow rate ranges are shown in FIGS. 3A and 3B.

Data transmission unit 208 transmits information stored in data storage unit 206 to controller 102 in electronic digital form. For example, communication unit 208 may thus be configured to transmit information related to the fluid delivery device 114 to controller 102 when the fluid outlet opening 202b of the connector 112 is connected to the fluid delivery device 114, and/or when the fluid outlet opening 202a of the connector 112 is connected to the nozzle connector 111 of the flow rate analyzer 110 or to the fluid regulator 106.

In an embodiment, where the connector 112 is not integrated with the fluid delivery device 114, once the connector 112 detects that the fluid outlet opening 202b of the connector 112 is connected to the fluid delivery device 114, communication unit 208 is triggered to transmit information related to fluid delivery device 114 to the controller 102 in digital electronic form. In another embodiment, where the connector 112 is integrated with the fluid delivery device 114, once the connector 112 detects that the fluid outlet opening 202a of the connector 112 is connected to the nozzle connector 111, communication unit 208 is triggered to transmit information related to fluid delivery device 114 to the controller 102. In another embodiment, the connector 112 detects that the fluid outlet opening 202b of the connector 112 is connected to the fluid delivery device 114 and the fluid outlet opening 202a of the connector 112 is connected to the nozzle connector 111, communication unit 208 is triggered to transmit information related to fluid delivery device 114 to the controller 102.

Connector 112 can detect and verify the connection between the fluid outlet opening 202a and the nozzle connector 111 and/or between the fluid outlet opening 202b of the connector 112 and the fluid delivery device 114 by a mechanical snap in system or switch, or the like. The connector 112 can also comprise an open/closed valve system that can be opened when the physical connection is made—this prevents waste of the fluid by spewing it into the room before a nozzle connector 111 connection or fluid delivery device 114 connection is made. The information can be transmitted via wired or wireless communication, such as Near Field Communication, for example, Bluetooth™, infrared, or RFID.

In the embodiment shown in FIG. 2, an RFID, either passive or active, performs the combined functions of data unit 206 and communication unit 208. Receiver 113 is configured to receive RFID signals, which carry information related to delivery device 114. If the RFID is passive, the RFID will be powered by the receiver 113 (RFID reader). If the RFID is active, the RFID has its own power source and does not need to be powered by the receiver 113 (RFID reader). Information stored in the communications data unit 206 is thus transmitted by the combined data/receiver unit to receiver 113 when the connector 112 detects that connector 112 has been connected with the fluid delivery device 114. The RFID reader is operatively connected to controller 102 to electronically transmit the received information to the controller 102 for further processing.

In one embodiment, information stored in data unit 206 identifies the type of connector 112; controller 102 stores relevant parameters associated with multiple types of fluid delivery device 114. When the identity of the selected type of delivery device 114 is transmitted from connector 112 to controller 102, controller 102 retrieves, from memory 122, relevant parameters associated with the selected delivery device 114, such as the flow rate range that is operational to the fluid delivery device 114, the flow rate that may be required to increase or decrease blood oxygen content by a specific amount and other parameters. In another embodiment, the information stored in the data unit 206 and transmitted to controller 102 relates to relevant parameters of device 114, including the flow rate range supported by delivery device, rather than simply identifying the type of the selected delivery device 114.

When controller 102 receives information from RFID receiver 113 in FIG. 2, controller 102 is thereby provided with information which identifies the type of selected delivery device 114. Pre-programmed information in controller 102 translates this identification information into parameters useful for regulating the gas flow rate to delivery device 114. Additional parameters relevant to device 114 may be pre-programmed into controller 102, such as whether the fluid delivery device 114 includes such functions as CO₂ sampling, interfacing with a humidifier or drug delivery capabilities.

In another embodiment, the functions of the connector 112 are integrated with the fluid delivery device 114 or tube 115 whereby the combined unit includes the data and communication units 206 and 208 as described above. In this aspect, connector 112 may comprise a conventional connector member configured to mate tube 115 to housing 109, and the data storage and communication functions described above are provided by electronic components integrated with the delivery device and/or tubing and/or integrated connector.

System 100 may also include one or more sensors 116, such as a pulse oximeter, for detecting the saturation level of a patient's blood. Sensor 116 can be of conventional design and can transmit the detected saturation level data via Bluetooth™ or other conventional wireless or wired electronic data transmission means. Sensor 116 detects and collects selected patient information, in digital or analogue form, such as the oxygen saturation level of the patient's blood, or SPaO2 in the blood of the patient. Other measured properties may include, but are not limited to, the patient's Pulse Rate (PR), inhalation/exhalation characteristics such as End Tidal Carbon Dioxide concentration (ETCO2), and Perfusion Index (PI), Total Hemoglobin (SpHe), Oxygen Content (SpOC™), Pleth Variability Index (PVI®), Methemoglobin (SpMet®), Carboxyhemoglobin (SpCO®), Acoustic Respiration Rate (RRa®). If the collected information is stored in analog form the analog signals can be converted to digital signals by an analog/digital converter which may be integrated with sensor 116 or controller 102. Sensor 116 electronically transmits the patient data to controller 102, which includes a receiver to receive the electronic information. Sensor 116 may be supplied with other components of the system as a kit, as shown in the example of FIG. 2B, or supplied separately, and may comprise a conventional unit having data transmission capabilities. Preferably, the information it transmitted in real-time as it is collected with minimal delay, so as to enable system 100 to operate in a rapid-response feedback mode.

In an embodiment, system 100 may the include one or more ETCO2 analyzers 117 for collecting and measuring exhaled gas samples received from delivery device 114. ETCO2 analyzer 117 may transmit the measured ETCO2 value to the controller 102.

System 100 optionally includes an input device 119 to receive input commands from an operator. Input device 119 transmits input data electronically to controller 102 to at least partially control the fluid flow rate. Input device 119 can comprise a conventional user interface such as a touch screen, adjustable knobs/buttons, mouse or a keyboard. The input commands can include, for example, the ability to set a threshold SpO2 level that triggers an alarm, various selectable operational modes, a target SpO2 level for the patient, stepwise increments for increasing or decreasing gas flow rates, frequencies for measuring the patient's SpO2 level or reading the gas flow rate from the flow rate analyzer 110, frequencies for comparing the measured SpO2 with the pre-set SpO2 level and adjust valve 108, etc.

System 100 optionally includes an output device 121 that displays various input commands as well as other system information such as the measured SpO2 level by the sensor 116, the measured ETCO2 level by the ETCO2 analyzer 117, the flow rate detected by the flow rate analyzer 110, the type of selected fluid delivery device 114 and the operational flow rate thereof. As well, output device 121 can display information collected and/or stored by the controller 102, such as the measured historical or real time gas flow rate and patient SpO2 levels. Output device may provide a visual display and/or printed output and/or communicate to a remote device for display on that device.

Input and output devices 119 and 121 may be integrated with housing 109 as a unit, or separate devices that communicate remotely with controller 102. Input and output devices 119 and 121 may comprise an “app” or other program that can be downloaded to a conventional smartphone, tablet etc.

System 100 further comprises a computer memory 122 to store information in digital electronic form, received by the controller 102 from various inputs thereto including connector 112, flow rate analyzer 110, sensor 116, ETCO2 analyzer 117, and input commands from input device 119. Memory 122 can store the information related to delivery devices 114, such as flow rate ranges supported by, or desired information related delivery devices 114. Memory 122 can store the information for a predetermined period, for example, 24 hours. Memory 122 can also store historical data such as adjustments that have been made during a selected period.

System 100 can comprise a data communication module, for example, a wireless communication module 123, such as a Wi-Fi module, to communicate data in electronic digital form to or from a central controller or database, such as an electronic medical records (EMR) database 126. Such data may include information that controller 102 has received from connector 112, current flow rate data from flow rate analyzer 110, patient SpO2 levels from sensor 116, patient ETCO2 levels from ETCO2 analyzer 117, and input commands from input device 119. In one embodiment, some or all of the functions of controller 102 are provided remotely via a remote computing device that communicates with system 100.

As well, system 100 can be updated remotely to accommodate different types of devices 114 that have not been previously programmed into controller 102.

With the uploaded information, a remote computing device can perform a wide variety of informational functions, for example to track the effectiveness of delivering oxygen during healthcare, calculate during a given period the actual oxygen administered at a fixed rate vs. the actual oxygen consumed, diagnose a medical condition that relates to the responsiveness of the patient to the fluid delivery, and suggest when an alternative delivery device should be used, or if the patient is entering into a state of distress.

System 100 can run on AC or DC current and may also have a battery back-up in case of a power failure.

System 100 can be set to operate either in an auto mode or a manual mode that overrides the auto mode, as described below.

### Auto Mode

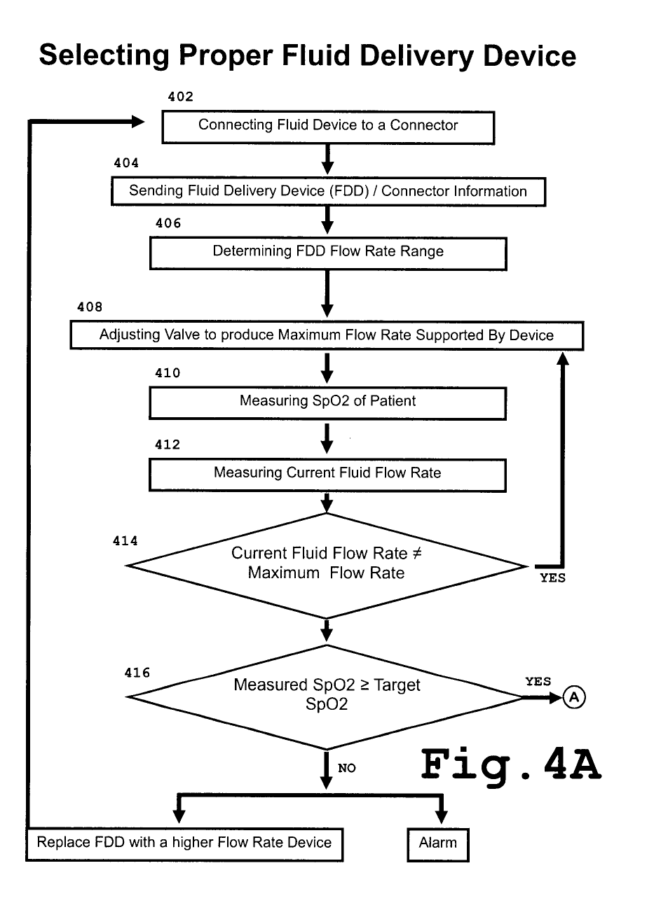

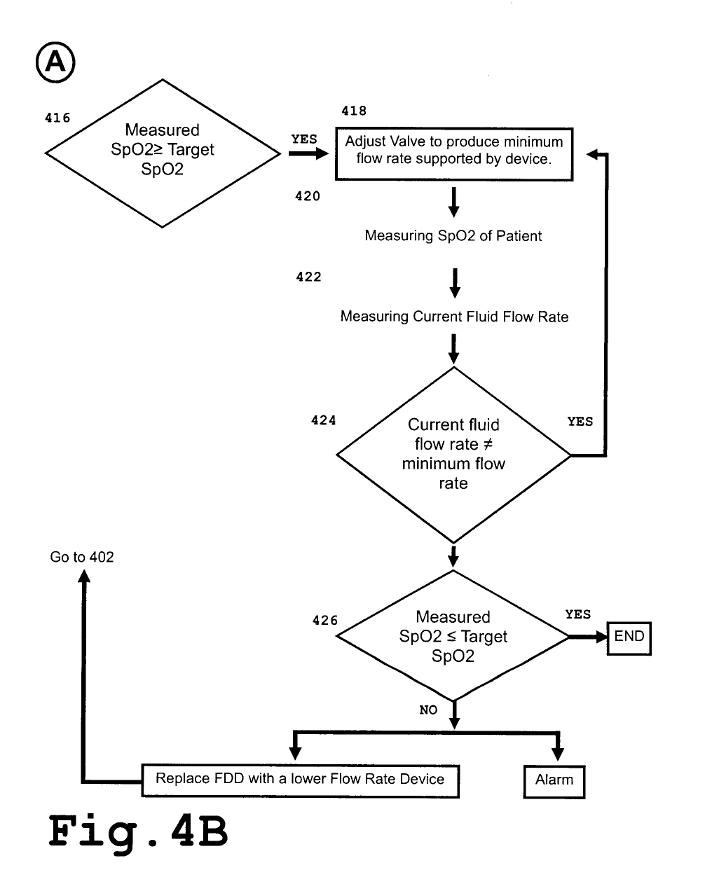

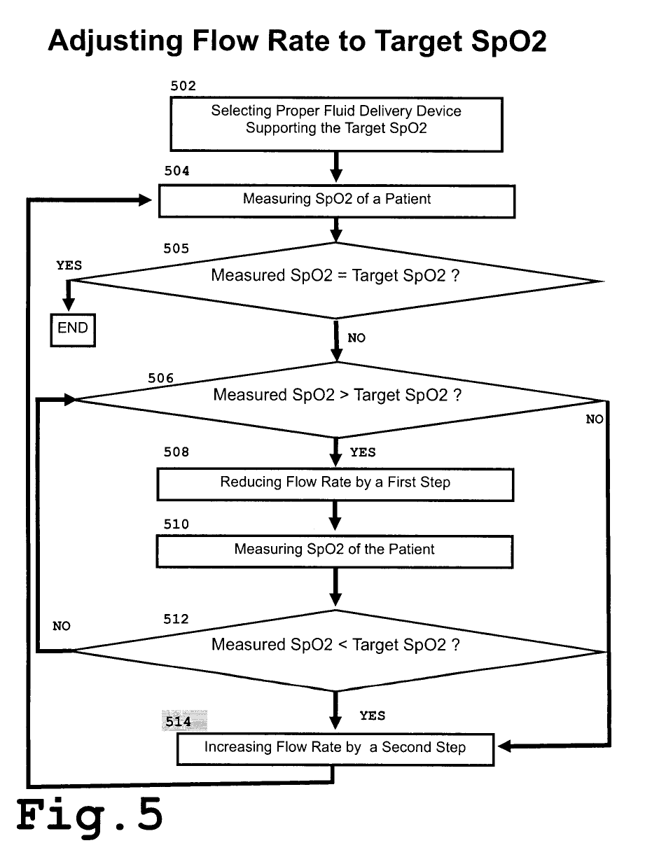

In auto mode, schematically shown in FIGS. 4 and 5, controller 102 automatically generates a feedback loop based on the level of a selected compound detected in the patient's blood, for example, SpO2 and/or ETCO2. The selected compound is one that will respond to the administration of a compound to the patient, which may be same or different as the selected compound in the patient's blood. In the present example, the selected compound in the patient's blood is oxygen, which, as is known, responds quickly to the administration of oxygen to the patient. In this example, the rate of oxygen flow from system 100 is adjusted to bring or maintain the patient oxygen levels, within a predetermined target range or a specific target level. For this purpose, controller 102 controls fluid regulator 106 to increase or decrease oxygen flow, within the operational flow rate range of fluid delivery device 114, in response to data received from oxygen sensor 116, or from ETCO2 analyzer 117.

At an initial set-up stage, connector 112 is connected to system 100 as described above, which triggers transmission of information to controller 102 reflective of information related to the selected device 114 connected thereto. Controller 102 is thereby “set” to operate under parameters specific to selected device 114, including its operational fluid flow range, expected physiological responses of a typical patient to specific flow rates, as well as other operating parameters. In response, controller 102 controls fluid regulator 106 to actuate valve 108 to provide a fluid flow rate that corresponds to an initial predetermined flow rate. This predetermined initial flow rate may be determined by controller 102 from information received from connector 112, for example a midpoint of the operational flow rate of device 114. In this example, the initial flow rate can be set based upon initial data from sensor 116 or ETCO2 analyzer 117, or alternatively based on a predetermined oxygen flow rate that is expected to maintain blood oxygen saturation at a desired level.

At a subsequent stage, controller 102 controls the gas flow rate in a recursive manner in response to inputs including detected compound levels, such as patient SpO2 levels from the sensor 116 and/or patient ETCO2 levels from ETCO2 analyzer 117, real time oxygen flow rate delivered to device 114, anticipated fraction of inspired oxygen (FiO2) from the patient and the predetermined target SpO2 or ETCO2. Controller 102 determines whether the flow rate range supported by selected delivery device 114 can provide the target SpO2/ETCO2. If so, controller 102 further regulates the fluid flow rate supplied to the patient to achieve the target SpO2/ETCO2. Controller 102 generates commands to adjust valve 108 based on the collected information.

According to one aspect, system 100 is used to select an appropriate delivery device 114 based on the responsiveness of the patient to various fluid delivery rates. In this example, shown in FIGS. 4A and 4B, system 100 performs the following steps to determine whether device 114 is capable of providing the target SpO2 in the patient:

a) At 402, fluid delivery device 114 (FDD) is connected, via connector 112, to system 100;

b) At 404, connector 112 transmits device information (device identification and/or device parameters) to controller 102;

c) At 406, controller 102 determines FDD flow rate range, for example, by retrieving from the memory 122 of controller 102 the flow rate range supported by selected device 114 or obtaining this information from connector 112;

d) At 408, controller 102 adjusts valve 108 of fluid regulator 106 to produce a maximum fluid flow rate supported by delivery device 114;

e) At 410, sensor 116 measures the SpO2 level in the blood of the patient;

f) at 412, flow rate analyzer 110 measures the current oxygen flow rate;

g) at 414, controller 102 may control fluid regulator 106 to further adjust valve 108 if the measured current fluid flow rate is different from the maximum oxygen flow rate;

h) controller 102 monitors the SpO2 level received from sensor 116 and the current fluid flow rate from flow rate analyzer 110;

i) at 416, controller 102 compares that the measured SpO2 to the predetermined target SpO2 level;

j) if the measured fluid flow rate has reached the maximum fluid flow rate of the flow rate range and if the measured compound level remains lower than the target level, replacing the fluid delivery device with a second fluid delivery device supporting a higher range of flow rate, and/or generating an alarm;

k) if the measured compound level exceeds or is equal to the target SpO2 level, at 418, At 408, controller 102 adjusts valve 108 of fluid regulator 106 to produce a minimum fluid flow rate supported by delivery device 114;

l) At 420, sensor 116 measures the SpO2 level in the blood of the patient;

m) at 422, flow rate analyzer 110 measures the current oxygen flow rate;

n) at 424, controller 102 may control fluid regulator 106 to further adjust valve 108 if the measured current fluid flow rate is different from the minimum fluid flow rate;

o) controller 102 monitors the SpO2 level received from sensor 116 and the current fluid flow rate from flow rate analyzer 110;

p) at 426, controller 102 compares that the measured SpO2 to the predetermined target SpO2 level;

q) if the measured compound level remains higher than the target level, replacing the fluid delivery device supporting a lower range of flow rate and/or generating an alarm; and

r) repeating steps a)-q) until the measured compound level less then or equal to the target level thereof.

The order of the steps may be varied. for example, step 410 may be performed after step 414 and before 416, and step 422 may be performed after step 424 and before 426.

Each flow rate adjustment can be performed continuously, after a predetermined time, or after different predetermined times for different steps.

If controller 102 detects that the gas flow rate is at the lower end of the operational range for device 114 but the detected patient SpO2 level is still higher than the target level (or the converse situation occurs), controller 102 triggers an alarm that indicates this information is communicated to an operator by, for example, a sound, visual signal and/or message transmitted to a remote device.

Controller 102 may optionally transmit information to an operator to suggest an alternative fluid delivery device 114 when the above alarms are triggered.

The example shown in FIGS. 4A and 4B also applies to determining whether device 114 is capable of providing a target ETCO2.

After a delivery device 114 that is capable of providing the required SpO2/ETCO2 level is selected, controller 102 regulates the fluid flow rate supplied to the patient via the selected delivery device 114 to achieve the target SpO2/ETCO2 level. In the example of FIG. 5, controller 102 regulates the fluid flow rate supplied to the patient via the selected delivery device 114 to achieve the target SpO2 level as follows:

a) At 502, select a delivery device 114 considered to be capable of supplying sufficient fluid to bring the patient to the target SpO2 level. This selection may be made using the method depicted in FIG. 4, or otherwise;

b) At 504, measure SpO2 of the patient;

c) At 505, determine whether the measured SpO2 is equal or substantially equal to the target SpO2; if not,

d) At 506, compare the measured SpO2 with the target SpO2;

e) If the measured SpO2 is greater than the target SpO2, at 508, reducing the flow rate by a first step, for example, 50%, if the measured SpO2 is higher than the predetermined target SPO2 level;

f) repeat steps a)-e) until the SpO2 is equal to or lower than the target SpO2 level;

g) at 510, measure SpO2 of the patient;

h) At 512, compare the measured SpO2 with the target SpO2, if the measured SpO2 is lower than the target SpO2 level, controller 102 at step 514 increases the flow rate by a second step, for example, 33%;

i) Controller 102 repeats step a)-g) until the measured SpO2 is equal or higher than the target SpO2; and

j) repeat steps b)-h) until the measured SpO2 is equal to or substantially equal to the target SpO2.

The reduction in flow rate generated in step d) generally exceeds the increase in flow in step g). The flow rate change of these respective steps may be varied. As well, each flow rate adjustment can be performed continuously, after a predetermined time, or after different predetermined times for different steps.

Controller 102, flow rate analyzer 110, connector 112, and sensor 116 form a closed data loop control to achieve a selected SpO2 level or oxygen flow rate.

The closed feedback loop control steps can be performed continuously or at a preset frequency, or performed intermittently at preset various frequencies for various steps.

In one embodiment, the preset target SpO2 is a physiological standard. The measured SpO2 from the pulse oximeter is compared to the physiological standard. If the measured SpO2 is lower than the physiological standard, controller 102 controls valve 108 to increase the flow rate. If the measured SpO2 is higher than the physiological standard, controller 102 controls valve 108 to decrease the flow rate.

Optionally, an operator can preset a range of SpO2 levels for a patient, whereby if the detected SpO2 falls outside this range, controller 102 will trigger an alarm.

Controller 102 may also regulate the fluid flow rate supplied to the patient via the selected delivery device 114 to achieve the target ETCO2 level in accordance with the example shown in FIG. 5.

### Manual Mode

A manual mode may be selected to override the auto mode in whole or part. In this mode, an operator manually adjusts valve 108 of fluid regulator 106 based on the information displayed on output device 121. Thus, controller 102 is bypassed for controlling fluid regulator 106, or at least the automatic operation thereof. In this mode, system 100 or fluid source 104 may have a simple display-type flowmeter that does not generate electronic signals, such as a floating ball-type flowmeter. The operator can manually adjust valve 108 based on the detected fluid flow rate

In manual mode, input device 119 receives input commands solely from an operator, for example to set a target flow rate. The controller 102 transmits these input commands to control fluid regulator 106, which in turn controls the operation of valve 108 to further control the flow rate or the volume of fluid supply within a given period.

Although the present disclosure describes methods and processes with steps in a certain order, one or more steps of the methods and processes may be omitted or altered as appropriate. One or more steps may take place in an order other than that in which they are described, as appropriate.

The scope of the invention should not be limited by specific embodiments or examples set forth herein but should be given the broadest interpretation consistent with the specification as a whole. The claims are not limited in scope to any preferred or exemplified embodiments of the invention.

1-32. (canceled)

33. An apparatus for communicating between a medical device, a sensor and a fluid delivery controller, wherein the medical device is for delivering a fluid to a patient, the sensor is for detecting the level of at least one compound within a patient and transmitting data relating to this level to the controller, and the controller is adapted to control fluid flow rate to the medical device, said apparatus comprising:
a body configured for connection to said medical device;
a data storage unit for storing digital information identifying said medical device and/or at least one property of said medical device; and
a data transmitting unit for electronically transmitting the stored information in the data storage unit to the controller.

34. The apparatus of claim 33 wherein said body comprises a conduit having a first end for direct or indirect connection to the controller, an opposing second end for direct or indirect connection to the medical device and a bore between the first and second ends for fluid flow through the conduit to the medical device.

35. The apparatus of claim 33 wherein said body is releasably connectable to said medical device.

36. The apparatus of claim 33 wherein said body is integrated with said medical device.

37. The apparatus of claim 33 further comprising at least one medical device configured for connection to the apparatus.

38. The apparatus of any one claim 33, wherein said property comprises an operational fluid flow rate range of said medical device.

39. The apparatus of claim 33, wherein the transmitting unit automatically transmits the stored information when the apparatus is operatively connected to the medical device and/or a source of the fluid.

40. The apparatus of claim 33, wherein the data storage unit and the transmitting unit are integrated within an RFID tag.

41. The apparatus of claim 33, wherein said medical device collects exhaled breath from the patient and said sensor comprises an End Tidal Carbon Dioxide concentration (ETCO2) analyzer for measuring CO2 concentration in the patient's breath.

42. A system for controlling a fluid flow rate delivering to a patient through a medical device, comprising:
a control unit comprising a controller and a fluid regulator for controlling the flow rate a fluid suitable for administration to a patient in response to electronic signals from said controller;
a data storage unit for storing digital information identifying said fluid delivery device and/or properties thereof;
a data transmitting unit for electronically transmitting the stored information in the data storage unit to the controller;
wherein said controller is configured to receive electronic signals from said data transmitting unit and from a sensor for detecting a level of at least one compound within said patient and to transmit a signal to said controller indicative of the detected level of said compound, wherein said controller is configured to process signals with a feedback loop to provide a recursive control over the delivery rate of said fluid to bring or maintain the level of said compound within the patient within a predetermined range or predetermined level.

43. The system of claim 42, wherein said data storage unit and data transmitting unit are integrated within a conduit having a first end for direct or indirect connection to the controller, an opposing second end for direct or indirect connection to the medical device and a bore between the first and second ends for fluid flow through the conduit to the medical device.

44. The system of claim 42 wherein said data storage unit and data transmitting unit are integrated within a body that is releasably connectable to said medical device or said data storage unit and data transmitting unit are integrated with said medical device.

45. The system of claim 42, further comprising at least one medical device configured for connection to the system.

46. The system of claim 42, wherein said properties include an operational flow rate range of said medical device.

47. The system of claim 42, wherein the transmitting unit automatically transmits the stored information when the apparatus is operatively connected to the fluid delivery device and/or a source of the fluid.

48. The system of claim 42, wherein the data storage unit and the transmitting unit are integrated within an RFID tag.

49. The system of claim 42 wherein said sensor comprises a pulse oximeter or an End Tidal Carbon Dioxide concentration (ETCO2) analyzer for measuring CO2 concentration in the patient's breath.

50. The system of claim 49 wherein said medical device collects exhaled breath from the patient for conveying to the sensor.

51. The system of claim 42 wherein said control unit further comprise a flow rate meter for measuring the flow rate delivered to said delivery device, and electronically transmitting the detected flow rate to the controller.

52. The system of claim 42 further comprising a user interface for communicating manually entered information to said controller.

53. The system of claim 42 wherein said controller activates an alarm when the detected level of the compound in the patient falls outside a predetermined range.

54. The system of claim 42 wherein the data storage and transmission units are integrated within an RFID tag.

55. The system of claim 42 further comprising at least one selected medical device configured for connection to said data storage unit and data transmitting unit.

56. The system of claim 42 further comprising said sensor.

57. A method for controlling the level of a compound in a patient, wherein the level of the compound is responsive to a fluid administered to the patient through a medical device, said method comprising the steps of:
transmitting a first datum from a data transmission unit to a controller identifying said medical device and/or properties thereof;
transmitting a second datum from a sensor to the controller indicating the level of the compound in the patient; and
in response to the first datum and the second datum, transmitting at least one command from a fluid regulator to the controller for the fluid regulator to control a fluid flow rate to be administered to the patient through the medical device, wherein the fluid flow rate controlled by the fluid regulator is within a range that is correlated to the first datum for generating a target level of the compound in the patient.

58. The method of claim 57, further comprising use of the apparatus of claim 10 for performing said method.

59. The method of claim 57, wherein said sensor comprises an End Tidal Carbon Dioxide concentration (ETCO2) analyzer for measuring CO2 concentration in the patient's breath and/or an oximeter.
